(19) United States
(12) Patent Application Publication
(10) Pub. No.: US 2026/0069170 A1
(43) Pub. Date: Mar. 12, 2026

(54) Blood Draw Device with Support Tube for Prevention of Kinking During Catheter Advancement

(71) Applicant: Becton, Dickinson and Company, Franklin Lakes, NJ (US)

(72) Inventors: Manish Kumar, Bengaluru (IN); Praveen Nalawade, Bangaluru (IN); Shishir Prasad, Ramsey, NJ (US); Karthik MR, Bangalore (IN)

(21) Appl. No.: 19/263,669

(22) Filed: Jul. 9, 2025

Related U.S. Application Data
(60) Provisional application No. 63/671,465, filed on Jul. 15, 2024.

(51) Int. Cl. A61B 5/15; A61M 39/02; A61M 39/10

(52) U.S. Cl. CPC A61B 5/1411; A61M 39/0208; A61M 39/1011; A61M 2039/0235; A61M 2205/0266; A61M 2205/3306; A61M 2205/3331; A61M 2205/3334

(57) ABSTRACT

A blood draw device for advancing an extension catheter into an indwelling catheter includes an introducer housing, a kink-resistant support tube, a primary advancement member, and a secondary advancement member. The primary advancement member is movable relative to the housing to move the catheter from a first catheter position within the introducer to a second catheter position where a catheter distal end extends out past the indwelling catheter. The secondary advancement member is distal from the primary advancement member and is movable relative to the housing to move the support tube from a first tube position within the introducer to a second tube position where a tube distal end extends out past the indwelling catheter. When in the second tube position, the support tube is arranged coaxially about a portion of the catheter, to prevent kinking thereof as it advances from the first catheter position to the second catheter position.

### CROSS-REFERENCE TO RELATED APPLICATION

The present application claims priority to United States Provisional Ser. No. 63/671,465 entitled “Blood Draw Device with Support Tube for Prevention of Kinking During Catheter Advancement” filed Jul. 15, 2024, the entire disclosure of which is hereby incorporated by reference.

### BACKGROUND OF THE INVENTION

### Field of the Invention

The present invention is directed to a blood draw device for introducing a catheter into an indwelling catheter of a vascular access device.

### Background of the Invention

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

Recent developments in the PIVC field have led to the emergence of technologies designed to facilitate blood draw using an in-dwelling PIVC and accompanying blood draw device. The main method by which these devices work is by employing an introducer for inserting a catheter, probe, tube, or other instrument through the catheter lumen of the PIVC, with the introducer being attached to a catheter adapter of the PIVC that provide for insertion of the catheter. For example, the catheter adapter may include a needleless access connector thereon by which the catheter may be introduced to provide access to the PIVC and into the patient's vasculature. A syringe and/or vacutainer may then be used collect blood samples without needing to subject the patient to additional needle sticks.

The introducer of a blood draw device typically includes a housing, a catheter movable within the housing so as to be extendable out therefrom for advancement into the in-dwelling PIVC, and an advancement member that may be actuated by an operator relative to the housing. That is, the advancement member may be moved distally by the operator to cause a corresponding movement of the catheter relative to the housing, such that the catheter may be advanced out from the distal end of the housing and into the in-dwelling PIVC. However, during deployment of the catheter of the blood draw device, especially when the distal end portion of the catheter reaches a curved region of the PIVC (near where the PIVC enters the skin) and is caused to curve back and forth as it passes along the vein, the catheter of the blood draw device is subject to a column load which can cause bending, kinking, and/or deformation of the catheter. As the catheter bends, it can move in random directions creating a sinusoidal wave, which then flattens against the sidewall of the housing of the blood draw device as the force increases. Additional force applied to the catheter by further movement of the advancement member can then cause the catheter to double back on itself and collapse—i.e., to “buckle.”

Accordingly, a need exists for a blood draw device and introducer thereof that provides for advancement of a catheter into the PIVC while minimizing the potential for bending, kinking, or deformation of the catheter. It is desirable for such kink prevention features to be easily incorporated into the blood draw device, such that the blood draw device may be manufactured at scale and at low cost.

### SUMMARY OF THE INVENTION

Provided herein is a blood draw device for advancing a catheter into an indwelling catheter of a vascular access device. The blood draw device includes an introducer comprising a housing having a proximal end portion and a distal end portion, with the housing defining an inner volume configured to movably receive the catheter. The blood draw device also includes a coupling member positioned at the distal end portion and coupleable to the vascular access device, a catheter movably received within the inner volume, and a support tube movably received within the inner volume and defining a tube lumen therein configured to receive the catheter, with the support tube formed of a flexible, kink-resistant material. The blood draw device further includes a primary advancement member movably coupled to the housing and configured to move relative to the housing to move the catheter between a first position, in which the catheter is disposed within the introducer or within the introducer and the coupling member, and a second position, in which a distal end portion of the catheter is disposed beyond the distal end portion of the introducer and into or distally past a distal end of the indwelling catheter. The blood draw device still further includes a secondary advancement member movably coupled to the housing, distal from the primary advancement member, and configured to move relative to the housing to move the support tube between a first tube position, in which the support tube is disposed within the introducer or within the introducer and the coupling member, and a second tube position, in which a distal end portion of the support tube is disposed beyond the distal end portion of the introducer and into or distally past a distal end of the indwelling catheter. With the support tube in the second tube position and the catheter moving from the first position to the second catheter position, the support tube is arranged coaxially about a portion of the catheter, to prevent kinking of the catheter as it is advanced from the first catheter position to the second catheter position.

In some embodiments, the support tube is formed of a shape memory alloy.

In some embodiments, the shape memory alloy comprises Nitinol.

In some embodiments, the primary advancement member and the secondary advancement member are arranged on the housing such that movement of the catheter from the first catheter position to the second catheter position, via distal movement of the primary advancement member along the housing, is enabled only after movement of the support tube from the first tube position to the second tube position, via distal movement of the secondary advancement member along the housing.

In some embodiments, the blood draw device includes further includes one or more catheter support inserts positioned within the inner volume and positioned between the primary advancement member and the secondary advancement member, each of the one or more catheter support inserts receiving the catheter to provide support to a length of the catheter that extends between the primary advancement member and the secondary advancement member.

In some embodiments, for a respective catheter support insert of the one or more catheter support inserts that is nearest the primary advancement member, the primary advancement member engages the catheter support insert during movement thereof when moving the catheter from the first catheter position to the second catheter position, with the primary advancement member engaging and moving the catheter support insert with respect to the housing.

In some embodiments, the one or more catheter support inserts comprises a plurality of catheter support inserts spaced apart along a length of the catheter between the primary advancement member and the secondary advancement member.

In some embodiments, the support tube comprises a sensor positioned at a distal end of the support tube and this is configured to acquire operational data when advancing the support tube from the first tube position to the second tube position.

In some embodiments, the sensor comprises a pressure sensor configured to evaluate a line pressure within the indwelling catheter, as the support tube moves from the first tube position to the second tube position.

In some embodiments, the sensor comprises a near infrared (NIR) sensor configured to provide positional data for the support tube relative to the indwelling catheter and/or to detect an occlusion within the indwelling catheter.

In some embodiments, the sensor comprises a flow sensor configured to detect a flow rate of fluid into the indwelling catheter.

In some embodiments, the flow sensor is housed on a split septum valve at the distal end of the support tube, the split septum valve allowing for passage of the catheter therethrough when the catheter is moved to second catheter position.

In some embodiments, the secondary advancement member includes an electronics module incorporated therein, the electronics module comprising a communication module operatively connected to the sensor to receive signals therefrom regarding the acquired operational data, an integrated circuit configured to process the signals received by the communication module, and an indicator controlled by the integrated circuit and configured to generate an audio or visual alert responsive to the acquired operational data.

In some embodiments, the support tube comprises scale markings thereon indicating a length by which the support tube has been distally advanced as it moves from the first tube position to the second tube position.

In some embodiments, each of the primary advancement member and the secondary advancement member comprises a first portion comprising an engagement portion movable along a top surface of the housing and a second portion positioned at least partially within the inner volume and joined to the first portion through a slot formed in the top surface of the housing; wherein for the primary advancement member, the second portion is coupled with a proximal end of the catheter, and wherein for the secondary advancement member, the second portion is coupled with a proximal end of the support tube.

In some embodiments, each of the primary advancement member and the secondary advancement member slide within the slot formed in the top surface of the housing, to move relative to the housing.

In some embodiments, the coupling device comprises a lock including: a coupler configured to engage the distal end portion of the housing; a first arm and a second arm configured to engage with the vascular access device or an adapter provided between the vascular access device and the lock; and a blunt cannula extending from the coupler and disposed between the first arm and the second arm, the blunt cannula configured to engage with the vascular access device or the adapter; wherein a lumen extends through the blunt cannula and the coupler, through which the support tube and catheter may be distally advanced.

Also provided herein is another blood draw device for advancing a catheter into an indwelling catheter of a vascular access device. The blood draw device includes an introducer comprising a housing having a proximal end portion and a distal end portion, with the housing defining an inner volume and with the housing including a slot formed therein. The blood draw device also includes a primary advancement member movably coupled to the housing and configured to slide within the slot of the housing and a secondary advancement member movably coupled to the housing and configured to slide within the slot of the housing, the secondary advancement member positioned distal from the primary advancement member. The blood draw device further includes a catheter movably received within the inner volume and comprising a distal end and a proximal end, with the proximal end secured to the primary advancement member such that movement of the primary advancement member causes a corresponding movement of the catheter, and a support tube movably received within the inner volume and defining a tube lumen therein configured to receive the catheter, the support tube comprising a distal end and a proximal end, with the proximal end secured to the secondary advancement member such that movement of the secondary advancement member causes a corresponding movement of the support tube. With each of the primary advancement member and the secondary advancement member moved to a respective distal-most position on the housing, a portion of each of the catheter and the support tube is disposed beyond the distal end portion of the introducer, with the catheter being advanced into and through the support tube to prevent kinking of the catheter as it is distally advanced.

In some embodiments, the support tube is formed of a shape memory alloy that resists kinking when the support tube is advanced distally into the indwelling catheter of the vascular access device, via movement of the secondary advancement member.

In some embodiments, the blood draw device further includes one or more catheter support inserts positioned within the inner volume and positioned between the primary advancement member and the secondary advancement member, each of the one or more catheter support inserts receiving the catheter to provide support to a length of the catheter that extends between the primary advancement member and the secondary advancement member.

### BRIEF DESCRIPTION OF THE DRAWINGS

FIG. 1 is a perspective view of a blood draw device, according to an embodiment of the disclosure;

FIG. 2 is a top view of the blood draw device illustrated in FIG. 1;

FIG. 3 is an exploded view of the blood draw device of FIG. 1;

FIG. 4 is a perspective view of a first member of a housing included in the blood draw device of FIG. 1;

FIG. 5 is a perspective view of a second member of the introducer housing included in the blood draw device of FIG. 1;

FIG. 6 is a rear perspective view of the introducer housing formed by coupling the first member illustrated in FIG. 4 to the second member illustrated in FIG. 5;

FIG. 7 is a front perspective view of the introducer housing illustrated in FIG. 6;

FIG. 8 is a cross-sectional view of the introducer taken along the line 8-8 in FIG. 6;

FIG. 9 is a rear perspective view of a lock included in the blood draw device of FIG. 1;

FIG. 10 is an exploded perspective view an extension catheter, a support tube, a connection tube, and an advancement member included in the blood draw device of FIG. 1;

FIG. 11A is a perspective view of the primary advancement member illustrated in FIG. 10;

FIG. 12A is a cross-sectional view of the blood draw device taken along the line 12A-12A in FIG. 2;

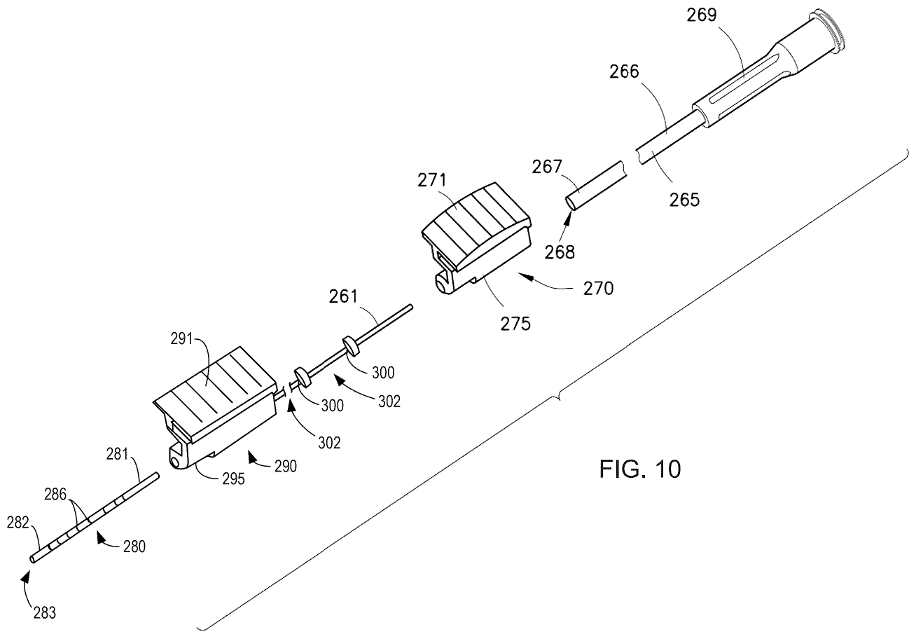

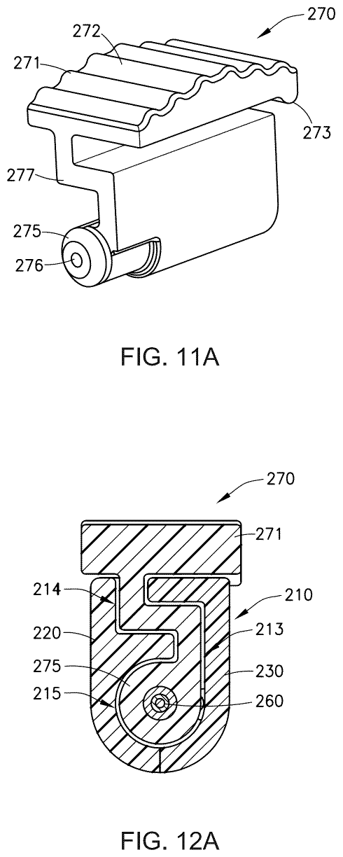

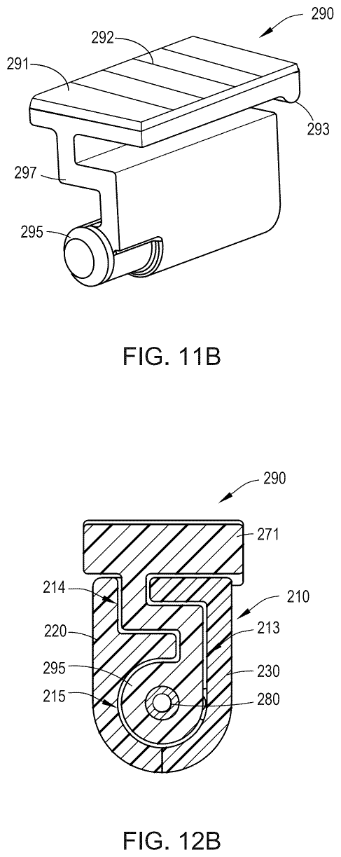

FIG. 11B is a perspective view of the secondary advancement member illustrated in FIG. 10;

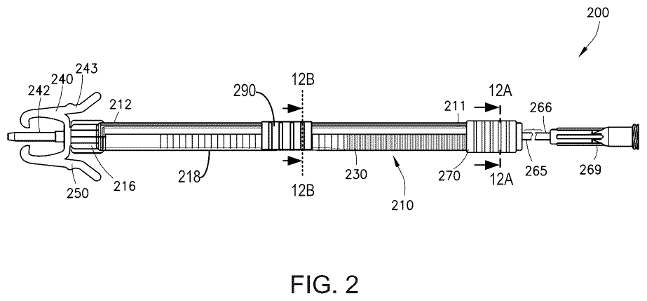

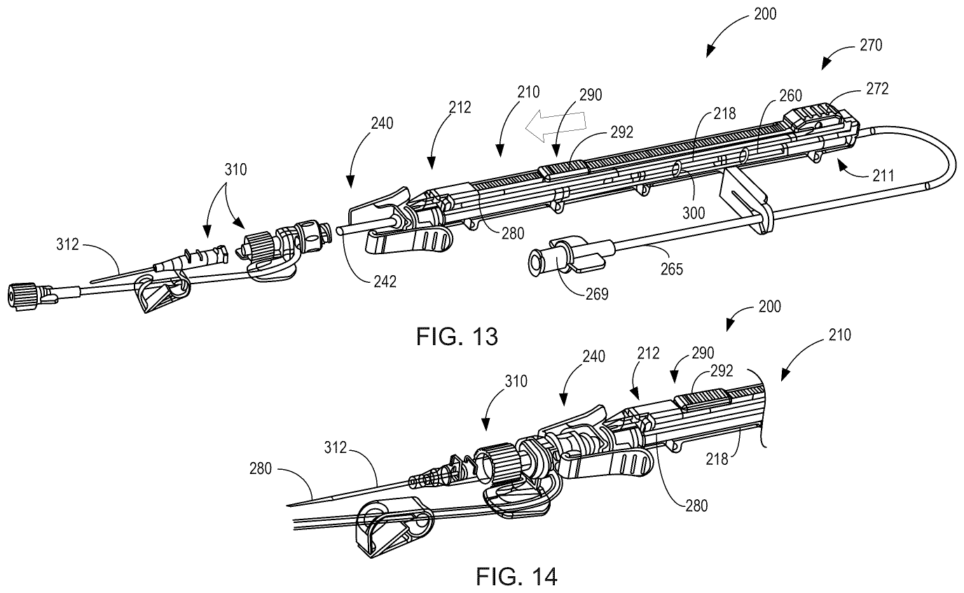

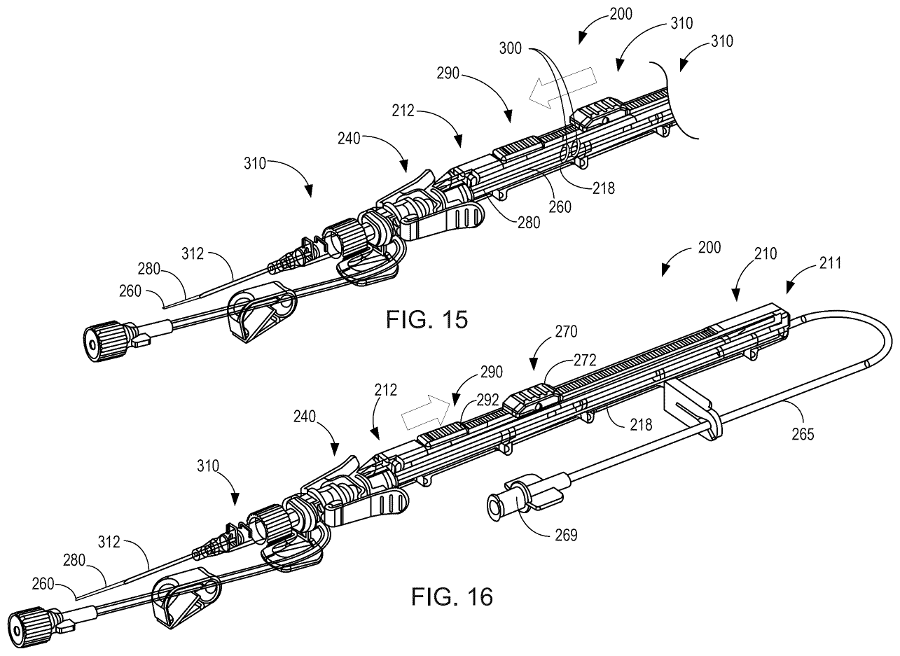

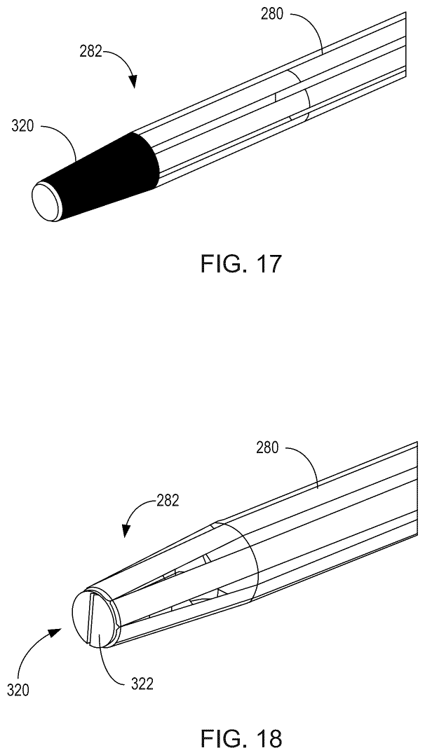

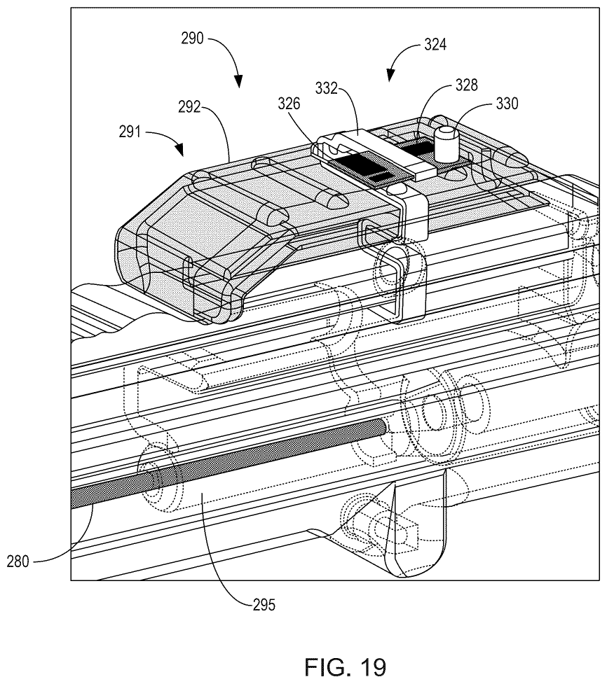

FIG. 12B is a cross-sectional view of the blood draw device taken along the line 12B-12B in FIG. 2;

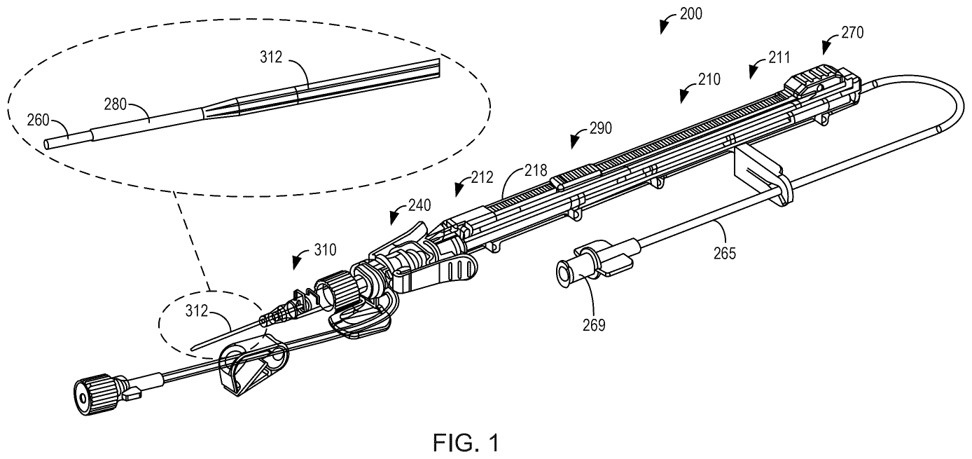

FIGS. 13-16 are perspective views of the blood draw device of FIG. 1 at various states when transitioning from a first configuration to a second configuration, showing positioning of the first and secondary advancement members, along with the extension catheter and support tube;

FIG. 17 is a detailed perspective view of the distal end portion of the support tube included in the blood draw device of FIG. 1, according to another embodiment of the disclosure;

FIG. 18 is a detailed perspective view of the distal end portion of the support tube included in the blood draw device of FIG. 1, according to another embodiment of the disclosure; and

FIG. 19 is a detailed perspective view of the secondary advancement member included in the blood draw device of FIG. 1, according to an embodiment of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

The present invention is directed to a blood draw device for phlebotomy through a peripheral intravenous line or catheter, with the blood draw device operable to advance an extension tube/catheter into the indwelling peripheral intravenous line and subsequently draw blood or administer a drug to a patient.

FIGS. 1-18 illustrate a blood transfer device 200 (i.e., blood draw device) and components thereof according to embodiments of the invention. The blood draw device 200, such as a PIVO® blood draw device, commercially available from Becton, Dickinson and Company, can be any suitable shape, size, or configuration and can be coupled to a vascular access device (VAD) 310, for example, via a lock and/or adapter. As described in further detail herein, a user can transition the blood draw device 200 from a first configuration to a second configuration to advance an extension catheter through an existing, placed, and/or indwelling catheter 312 of the VAD 310 (i.e., when the blood draw device 200 is coupled to the VAD) such that at least an end portion of the extension catheter is disposed in a distal position relative to the indwelling catheter 312. Moreover, with indwelling catheters each having a shape, size, and/or configuration that can vary based on, for example, a manufacturer of the VAD 310 and/or its intended usage, the blood draw device 200 can be arranged to allow the blood draw device 200 to be coupled to a VAD 310 having any suitable configuration and subsequently, to advance at least a portion of a catheter through the indwelling catheter 213. In addition, the blood draw device 200 can be manipulated by a user to place a distal surface of the extension catheter a predetermined and/or desired distance beyond a distal end of the indwelling catheter 312 to be disposed within a portion of a vein that receives a substantially unobstructed flow of blood.

As shown in FIGS. 1-3, the blood draw device 200 includes an introducer 210, a coupling member or lock 240 (hereafter “lock 240”), an extension catheter 260 (hereafter, “catheter 260”), a connection tube 265, a primary advancement member 270, a support tube 280, and a secondary advancement member 290. The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the blood draw device 200, which in some instances, can allow a user to manipulate the blood draw device 200 with one hand (i.e., single-handed use).

As shown in FIGS. 2-8, the introducer 210 of the blood draw device 200 includes a housing 218 having a first housing member 220 and a second housing member 230 that are coupled to collectively form the housing 218. The first housing member 220 includes a proximal end portion 221, a distal end portion 222, and an inner surface 223. The inner surface 223 has a first portion 224 and a second portion 225, with second portion 225 being a flange in one embodiment. The proximal end portion 221 of the first housing member 220, and more specifically, a proximal wall of the first housing member 220 defines a notch 226 configured to selectively receive a portion of the connection tube 265, as described in further detail herein.

The second housing member 230 has a proximal end portion 231, a distal end portion 232, an inner surface 233, and a top surface 235. As described above with reference to the first housing member 220, the proximal end portion 231 of the second housing member 230, and more specifically, a proximal wall of the second housing member 230 defines a notch 234 configured to selectively receive a portion of the connection tube 265.

The top surface 235 of the second housing member 230 includes a set of ribs 236 distributed along a length of the second housing member 230, with each rib 236 extending along a width of the second housing member 230. The ribs 236 formed by the top surface 235 of the second housing member 230 can be any suitable shape, size, and/or configuration. For example, the set of ribs 236 includes a first portion 237 having a first size and shape, and a second portion 238 having a second size and shape, different from the first size and shape. Each rib in the second portion 238 has a size and shape that is greater than the size and shape of each rib of the first portion 237.

As described in further detail herein, a portion of the advancement member 270 is configured to be advanced along the top surface 235 forming the set of ribs 236 as a user moves the advancement member 270 relative to the housing 218, which in turn, vibrates the advancement member 270 (and the catheter 260 coupled thereto). In some instances, this vibration can, for example, facilitate the advancing of the catheter 260 through a portion of the blood draw device 200, a portion of the VAD 310, and/or a portion of the vasculature. In some instances, the greater size of the ribs of the second portion 238 can result in a larger amount of vibration as the advancement member 270 is moved along the top surface 235 (as described above). In some instances, the greater size of the ribs of the second portion 238 can result in an increase in a force otherwise sufficient to move the portion of the advancement member 270 along the top surface 235.

Similar to primary advancement member 270, a portion of the secondary advancement member 290 is configured to be advanced along the top surface 235 forming the set of ribs 236 as a user moves the secondary advancement member 290 relative to the housing 218, which in turn, vibrates the secondary advancement member 290. In some instances, this vibration can, for example, facilitate the advancing of the support tube 280 coupled to (secondary advancement member 290) through a portion or the blood draw device 200, a portion of the VAD 310, and/or a portion of the vasculature.

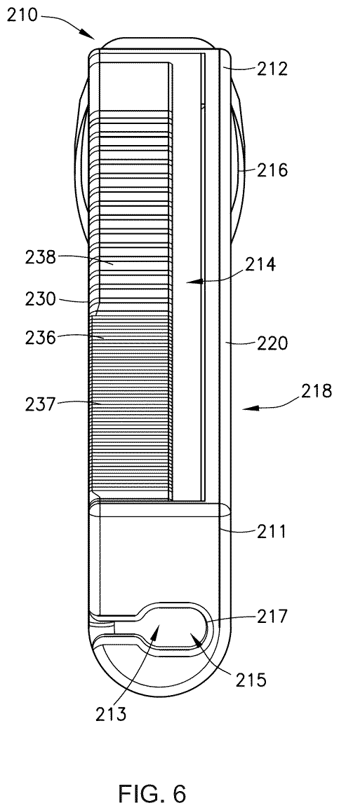

As shown in FIGS. 6-8, the first housing member 220 is configured to be coupled to the second housing member 230 to collectively form the housing 218. In some embodiments, forming the housing 218 from the first housing member 220 and the second housing member 230 can reduce undesirable variations in the shape and/or size of the inner surface 223 and 233 (e.g., due to draft angles and/or manufacturing tolerances) during manufacturing, which in some instances, can reduce a likelihood of kinks, bends, and/or deformations of the catheter 260 during use of the blood draw device 200, In some embodiments, forming the housing 218 from the first housing member 220 and the second housing member 230 can allow at least the inner surface 223 of the first housing member 220 to form a tortuous shape that would otherwise present challenges when manufacturing the housing 218 from a single work piece.

The first housing member 220 and the second housing member 230 collectively form a proximal end portion 211 and a distal end portion 212 of the housing 218 and collectively define an inner volume 213 of the housing 218. As shown in FIG. 6, the proximal end portion 211 of the housing 218 defines an opening 217. Specifically, the opening 217 is collectively formed and/or defined by the notch 226 of the first housing member 220 and the notch 234 of the second housing member 230. The arrangement of the proximal end portion 211 is such that a portion of the opening 217 defined by the notch 226 of the first housing member 220 has a first size and/or shape and a portion of the opening 217 defined by the notch 234 of the second housing member 230 has a second size and/or shape that is less than the first size and/or shape. In other words, a portion of the opening 217 is constricted, pinched, obstructed, and/or otherwise reduced. As described in further detail herein, the opening 217 is configured to receive a portion of the connection tube 265, which can be moved within the opening 217 from the larger portion of the opening 217 to the reduced portion of the opening 217 (e.g., the portion formed by the notch 234 of the second housing member 230) to obstruct, pinch, and/or clamp the connection tube 265.

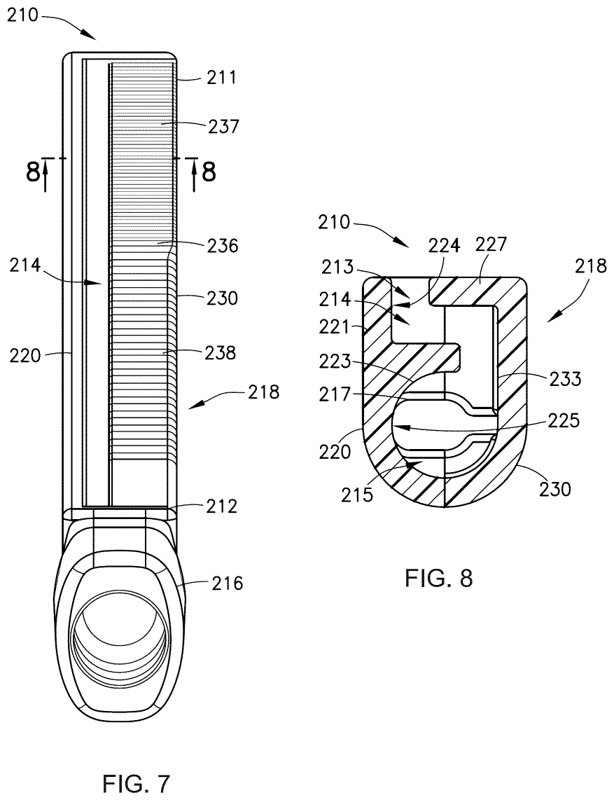

As shown in FIG. 7, the distal end portion 212 of the housing 218 includes and/or otherwise forms a coupler 216. In other words, the distal end portion 222 of the first housing member 220 and the distal end portion 232 of the second housing member 230 collectively form the coupler 216 at the distal end portion 212 of the housing 218. The coupler 216 can be any suitable shape, size, and/or configuration. For example, in this embodiment, the coupler 216 forms a set of threads, which can form a threaded coupling with an associated threaded portion of the lock 240, as described in further detail herein, Although not shown in FIG. 7, the distal end portion 211 of the housing 218 can include and/or can be configured to receive a seal (not shown) that can selectively seal and/or fluidically isolate the inner volume 213 of the housing 218 (at least from an open portion of the coupler 216). In use, the seal can be transitioned from a sealed or closed configuration to an open configuration to allow, for example, a portion of the support tube 280 and a portion of the catheter 260 to pass therethrough. In some embodiments, the seal can contact an outer surface of the support tube 280 to define a seal therebetween that is operable to limit and/or substantially prevent a back flow of fluid between the outer surface of the cannula and the seal.

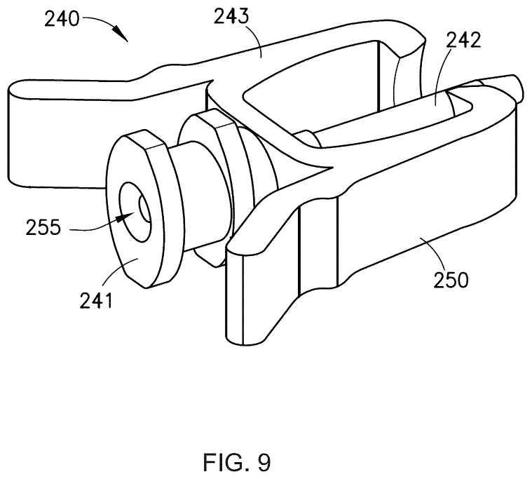

The inner surface 223 of the first housing member 220 and the inner surface 233 of the second housing member 230 collectively define the inner volume 213 of the housing 218. As shown in FIG. 8, the arrangement of the inner surfaces 223 and 233 is such that the inner volume 213 has and/or defines a tortuous cross-sectional shape. For example, the inner volume 213 can have a substantially S-shaped or an at least partially S-shaped cross-sectional shape. More specifically, the inner surface 223 of the first housing member 220 includes and/or forms a ridge, tab, flange, protrusion, and/or wall configured to separate the first portion 224 of the inner surface 223 from the second portion 225 of the inner surface 223. Thus, the tortuous cross-sectional shape of the inner volume 213 forms and/or defines a first portion 214 of the inner volume 213 and a second portion 215 of the inner volume 213. In this manner, the first portion 214 of the inner volume 213 is spaced apart from the second portion 215 of the inner volume 213 without being fluidically isolated therefrom. In other words, the first portion 214 of the inner volume 213 defines an axis that is parallel to and offset from an axis defined by the second portion 215 of the inner volume 213.

As shown in FIG. 8, the first portion 214 of the inner volume 213 extends through a wall of the housing 218. Similarly stated, the housing 218 defines (e.g., the first housing member 220 and the second housing member 230 collectively define) a slot, channel, track, opening, and/or the like that is in fluid communication with the first portion 214 of the inner volume 213. Conversely, the second portion 215 of the inner volume 213 is entirely defined and/or enclosed (at least in the circumferential direction) by the housing 218. The tortuous cross-sectional shape of the inner volume 213 is such that the second portion 215 cannot be viewed (e.g., is out of the line of sight) via the slot (in fluid communication with the first portion 214 of the inner volume 213), which in turn, can limit and/or substantially prevent contamination of the catheter 260 and support tube 280 disposed therein.

In this embodiment, the second portion 215 of the inner volume 213 is substantially aligned with, for example, a portion of the opening 217 and a portion of an opening defined by the coupler 216. Moreover, the second portion 215 of the inner volume 213 is configured to be substantially aligned with the lock 240 when the lock is coupled to the coupler 216 of the housing 218. In other words, the axis defined by the second portion 215 of the inner volume 213 is substantially co-axial with an axis defined by a portion of the lock 240, as described in further detail herein. In this manner, the second portion 215 of the inner volume 213 can movably receive, for example, a portion of the advancement member 270 and a portion of the catheter 260, as well as a portion of the advancement member 290 and a portion of the support tube 280. Thus, the advancement members 270, 290 can be moved relative to the housing 218 to (1) move the catheter 260 between a first catheter position, in which the catheter 260 is entirely disposed within the second portion 215 of the inner volume 213, and a second catheter position, in which at least a portion of the catheter 260 extends outside of the second portion 215 of the inner volume 213 and distal to the housing 218, and (2) move the support tube 280 between a first tube position, in which the tube 280 is entirely disposed within the second portion 215 of the inner volume 213, and a second tube position, in which at least a portion of the support tube 280 extends outside of the second portion 215 of the inner volume 213 and distal to the housing 218, as described in further detail herein.

The lock 240 of the blood draw device 200 can be any suitable shape, size, and/or configuration. As described above, the lock 240 is configured to be physically and fluidically coupled to the housing 218 and configured to couple the introducer 210 to the VAD 310 and/or any suitable intermediate device or adapter coupled to the VAD 310. The lock 240 has a coupler 241, a blunt cannula 242, a first arm 243, and a second arm 250, as shown in FIG. 9. In addition, the lock 240 defines a lumen 255 extending through the coupler 241 and the blunt cannula 242. The coupler 241 is configured to couple the lock 240 to the coupler 216 of the housing 218. Specifically, in this embodiment, the coupler 241 includes and/or forms one or more protrusions configured to selectively engage the threads defined and/or formed by the coupler 216 of the housing 218, thereby forming a threaded coupling.

The blunt cannula 242 extends from the coupler 246 and is disposed between the first arm 243 and the second arm 250. The blunt cannula 242 can be any suitable shape, size, and/or configuration. In some embodiments, the blunt cannula 242 can have a length that is sufficient to extend through at least a portion of the VAD 310 or through an adapter and at least partially into or through the VAD 310. Furthermore, the blunt cannula 242 can have an inner diameter (a diameter of a surface at least partially defining the lumen 255) that is similar to or slightly larger than an outer diameter of a portion of the support tube 280 and catheter 260. Thus, the lumen 255 of the lock 240 can receive a portion of the support tube 280 and catheter 260 when the blood draw device 200 is transitioned between the first configuration and the second configuration.

As described above, at least a portion of the catheter 260, support tube 280, and connection tube 265 are movably disposed within the second portion 215 of the inner volume 213 defined by the housing 218.

As shown in FIG. 10, the catheter 260 has a proximal end portion 261 and a distal end portion 262 and defines a lumen 263. The proximal end portion 261 of the catheter 260 is coupled to a second portion 275 of the advancement member 270. In this manner, the advancement member 270 can be moved relative to the housing 218 to move the catheter 260 between a first position, in which the catheter 260 is disposed within the housing 218 (e.g., the entire catheter 260 is disposed within the housing 218 or within the housing 218 and the lock 240) and a second position, in which the distal end portion of the catheter 260 is at least partially disposed in a position distal to the lock 240 and distal to the distal end of the indwelling catheter 312 (see FIG. 1) when the lock 240 is coupled to the VAD 310, as described in further detail herein. The distal end portion 262 can be any suitable shape, size, and/or configuration and can define at least one opening in fluid communication the lumen 263.

The connection tube 265 has a proximal end portion 266 and a distal end portion 267 and defines a lumen 268. A portion of the connection tube 265 is disposed within and extends through the opening 217 of the housing 218 (e.g., collectively defined by the notches 223 and 233 of the first housing member 220 and second housing member 230, respectively). As such, the proximal end portion 266 is at least partially disposed outside of the housing 218 and the distal end portion 267 is at least partially disposed within the second portion 215 of the inner volume 213 defined by the housing 218. As described above, the connection tube 265 can be moved within the opening 217 between a first position and a second position to selectively clamp, pinch, kink, bend, and/or otherwise deform a portion of the connection tube 265, which in turn, obstructs, pinches, kinks, closes, seals, etc. the lumen 268 of the connection tube 265. For example, the first position can be associated and/or aligned with a first portion of the opening 217 having a larger perimeter and/or diameter than a perimeter and/or diameter of a second portion of the opening 217 associated and/or aligned with the second position. Thus, a user can manipulate the connection tube 265 to occlude the lumen 268 of the connection tube 265, thereby limiting, restricting, and/or substantially preventing a flow of a fluid therethrough.

As shown in FIG. 10, the proximal end portion 266 of the connection tube 265 is coupled to and/or otherwise includes a coupler 269. The coupler 269 is configured to physically and fluidically couple the connection tube 265 to any suitable device such as, for example, a fluid reservoir, fluid source, syringe, evacuated container holder (e.g., having a sheathed needle or configured to be coupled to a sheathed needle), pump, and/or the like. The distal end portion 267 of the connection tube 265 is at least partially disposed within the second portion 215 of the inner volume 213 defined by the housing 218 and is coupled to a second portion 275 of the advancement member 270. In some embodiments, the connection tube 265 can have a larger diameter than the catheter 260 such that the proximal end portion 261 of the catheter 260 is at least partially disposed within the lumen 268 defined by the connection tube 265 when the catheter 260 and the connection tube 265 are coupled to the second portion 275 of the advancement member 270, In some embodiments, such an arrangement can, for example, reduce and/or substantially prevent leaks associated with fluid flowing between the catheter 260 and the connection tube 265. In some embodiments, such an arrangement can also limit, reduce, and/or substantially prevent hemolysis of a volume of blood as the volume of blood flows through the catheter 260 and the connection tube 265. In this manner, when the coupler 269 is coupled to a fluid reservoir, fluid source, syringe, evacuated container, pump, etc., the connection tube 265 establishes fluid communication between the reservoir, source, pump, etc. and the catheter 260.

As further shown in FIG. 10, the support tube 280 is provided in blood draw device 200 at a location that is distal from the secondary advancement member 290. The support tube 280 has a proximal end portion 281 and a distal end portion 282 and defines a lumen 283. The proximal end portion 281 of the support tube 280 is coupled to a second portion 295 of the advancement member 290. In this manner, the advancement member 290 can be moved relative to the housing 218 to move the support tube 280 between a first position, in which the support tube 280 is disposed within the housing 218 (e.g., the entire support tube 280 is disposed within the housing 218 or within the housing 218 and the lock 240) and a second position, in which the distal end portion of the support tube 280 is disposed in a position distal to the lock 240 and distal to the distal end of the indwelling catheter 312 when the lock 240 is coupled to the VAD 310, as described in further detail herein. The distal end portion 282 can be any suitable shape, size, and/or configuration and can define at least one opening in fluid communication the lumen 283.

According to embodiments of the disclosure, the support tube 280 has a larger diameter than the catheter 260 (i.e., an inner diameter of lumen 283 is larger than an outer diameter of catheter 260), such that the distal end portion 262 of the catheter 260 may be at least partially disposed within the lumen 283 defined by the support tube 280 when the catheter is moved to the second position—i.e., the support tube 280 is positioned coaxially about the catheter 260. In operation of the blood draw device 200, the support tube 280 helps in guiding the catheter 260 to the second catheter position (past the tip of the indwelling catheter 312), with the support tube 280 preventing kinking of the catheter 260 as it is advanced distally through the VAD 310 and into the vasculature of a patient.

According to exemplary embodiments, in order to allow for advancement of the catheter 260 from the first catheter position to the second catheter position, the support tube 280 is made of a flexible material having anti-kinking properties. Such material may comprise a shape memory alloy, such as Nitinol as a non-limiting example. With the support tube 280 formed from an anti-kinking material, the support tube 280 is able to guide itself through the VAD 310 (and indwelling catheter 312) as it is advanced from the first tube position to the second tube position, even if the indwelling catheter 312 has bends where it enters the vein from the skin surface. With the support tube 280 advanced to the second tube position without any kinks therein, the catheter 260 may then subsequently be moved to its second catheter position—with the catheter 260 advancing through the support tube 280 and with the support tube 280 prevent kinking of the catheter 260 as it is advanced therethrough, due to the coaxial arrangement of the support tube 280 about the catheter 260.

In some embodiments, and as shown in FIG. 10, the support tube 280 may include scale markings 286 thereon that allow a clinician or other user to estimate the location of the support tube 280 with respect to a distal tip of the indwelling catheter 312 when the support tube 280 is moved to its second tube position. That is, via the scale markings 286 provided on the support tube 280, a user may be able to determine when the support tube 280 is placed at a desired location within the vasculature (and relative to the indwelling catheter 312) from where fresh blood can be drawn. The scale markings 286 may be visible through the housing 218 of introducer 210, to provide for such location determination.

In operation of the blood draw device 200, each of the primary advancement member 270 and the secondary advancement member 290 of the blood draw device 200 can be moved along a length of the housing 218 to transition the blood draw device 200 between its first configuration, in which the catheter 260 is in the first catheter position and the support tube 280 is in the first tube position, and its second configuration, in which the catheter 260 is in the second catheter position and the support tube 280 is in the second tube position. The advancement members 270, 290 can be any suitable shape, size, and/or configuration. For example, in some embodiments, the advancement members 270, 290 can have a size and shape that is associated with and/or based at least in part on a size and/or shape of the housing 218.

Referring now to FIGS. 11A and 11B and FIGS. 12A and 12B, the configuration of the primary advancement member 270 and the secondary advancement member 290, along with positioning thereof within housing 218 is shown and described in more detail.

As shown in FIGS. 11A and 12A, the primary advancement member 270 includes a first portion 271, the second portion 275, and a wall 277 extending therebetween, The first portion 271 of the advancement member 270 is at least partially disposed within the first portion 214 of the inner volume 213 defined by the housing 218 and the second portion 275 of the advancement member 270 is disposed within the second portion 215 of the inner volume 213, as described above. The first portion 271 of the advancement member 270 includes an engagement member 272. The arrangement of the advancement member 270 is such that the engagement member 272 is disposed outside of the housing 218 while the rest of the first portion 271 is within the first portion 214 of the inner volume 213 defined by the housing 218. As such, the engagement member 272 can be engaged and/or manipulated by a user (e.g., by a finger or thumb of the user) to move the advancement member 270 relative to the housing 218. In some embodiments, the engagement member 272 can include a set of ridges and/or any suitable surface finish that can, for example, increase the ergonomics of the advancement member 270 and/or blood draw device 200.

The engagement member 272 includes a tab 273 disposed at or near a proximal end portion of the engagement member 272. The tab 273 can be any suitable tab, rail, ridge, bump, protrusion, knob, roller, slider, etc. that extends from a surface of the engagement member 272. The tab 273 is configured to selectively engage the top surface 235 of the second housing member 230 of the housing 218. More specifically, the tab 273 is in contact with the ribs 236 formed by the second housing member 230 and moves along each successive rib as the advancement member 270 is moved along a length of the housing 218, with interaction of the tab 273 with the ribs 236 causing vibration of the advancement member 270.

The second portion 275 of the primary advancement member 270 includes an opening 276 formed therein with which catheter 260 can be inserted/engaged, to secure the proximal end 261 of the catheter 260 to the advancement member 270.

As shown in FIGS. 11B and 12B, the secondary advancement member 290 includes a first portion 291, a second portion 295, and a wall 297 extending therebetween. The first portion 291 of the advancement member 290 is at least partially disposed within the first portion 214 of the inner volume 213 defined by the housing 218 and the second portion 295 of the advancement member 290 is disposed within the second portion 215 of the inner volume 213, as described above. The first portion 291 of the advancement member 290 includes an engagement member 292. The arrangement of the advancement member 290 is such that the engagement member 292 is disposed outside of the housing 218 while the rest of the first portion 291 is within the first portion 214 of the inner volume 213 defined by the housing 218. As such, the engagement member 292 can be engaged and/or manipulated by a user (e.g., by a finger or thumb of the user) to move the advancement member 290 relative to the housing 218. In some embodiments, the engagement member 292 can include a set of ridges and/or any suitable surface finish that can, for example, increase the ergonomics of the advancement member 290 and/or blood draw device 200.

The engagement member 292 includes a tab 293 disposed at or near a proximal end portion of the engagement member 292. The tab 293 can be any suitable tab, rail, ridge, bump, protrusion, knob, roller, slider, etc. that extends from a surface of the engagement member 292. The tab 293 is configured to selectively engage the top surface 235 of the second housing member 230 of the housing 218. More specifically, the tab 293 is in contact with the ribs 236 formed by the second housing member 230 and moves along each successive rib as the advancement member 290 is moved along a length of the housing 218, with interaction of the tab 293 with the ribs 236 causing vibration of the advancement member 290.

The second portion 295 of the secondary advancement member 290 includes an opening 296 formed therein with which support 280 can be inserted/engaged, to secure the proximal end 281 of the support tube 280 to the advancement member 290. Additionally, the opening 296 allows for the catheter 260 to be advanced through the secondary advancement member 290 when the catheter 260 is moved from the first catheter position to the second catheter position.

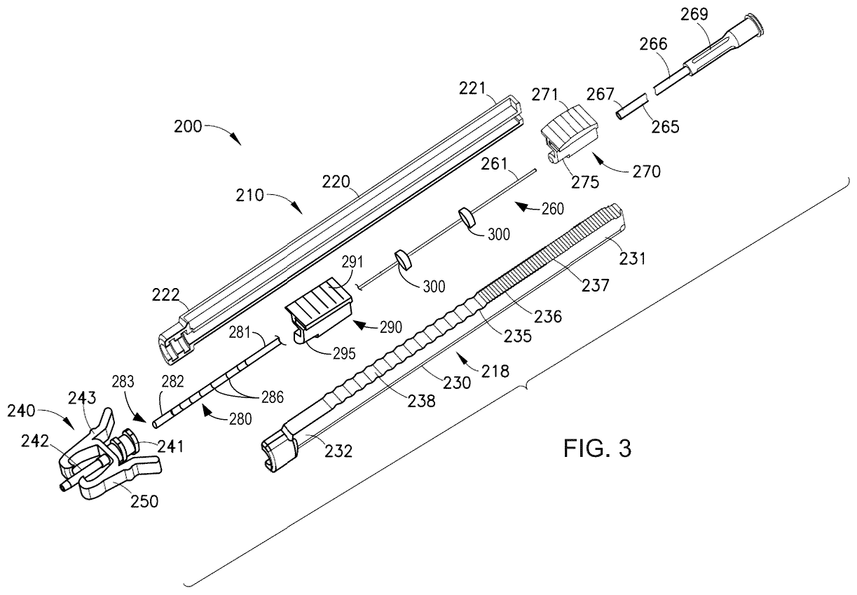

As shown in FIGS. 3 and 10, in some embodiments of the disclosure, one or more catheter support inserts 300 (or, more generally, “support inserts”) may optionally be included in introducer 210 and positioned along a length of the catheter 260 between the proximal and distal ends thereof. As shown in FIGS. 3 and 10, two support inserts 300 are positioned along a length of the catheter 260, but it is recognized a lesser or greater number of support inserts 300 could be provided in introducer-including only a single support insert 300 positioned on catheter 260, or two, three, five, or more support inserts 300 potentially being positioned along a length of the catheter 260. With multiple support inserts 300 being provided, the support inserts 300 may be spaced equidistantly along the catheter 260.

The support inserts 300 of introducer 210 are positioned within the housing 218 (i.e., within the second portion 215 of the inner volume 213) at locations between the primary advancement member 270 and the secondary advancement member 290. The catheter 260 is routed through a passageway or opening 302 of each of the support inserts 300 to reduce an unsupported length of the catheter 260, effectively increasing the resistance the catheter 260 can withstand before buckling or kinking. That is, the force at which a catheter 260 buckles/kinks is inversely proportional to the effective length squared. Thus, as the effective length of the catheter 260 increases, the force required to buckle/kink the catheter 260 decreases, making the catheter more prone to buckling/kinking. The support inserts 300 support the catheter 260, thereby shortening the effective length of the catheter 260, increasing the force required to buckle/kink the catheter 260, and decreasing the tendency of the catheter 260 within housing 218 to buckle/kink—i.e., the support inserts 300 divide the unsupported length of the catheter 260 into smaller portions that are more resistant to bending, kinking, flexing, and/or deformation.

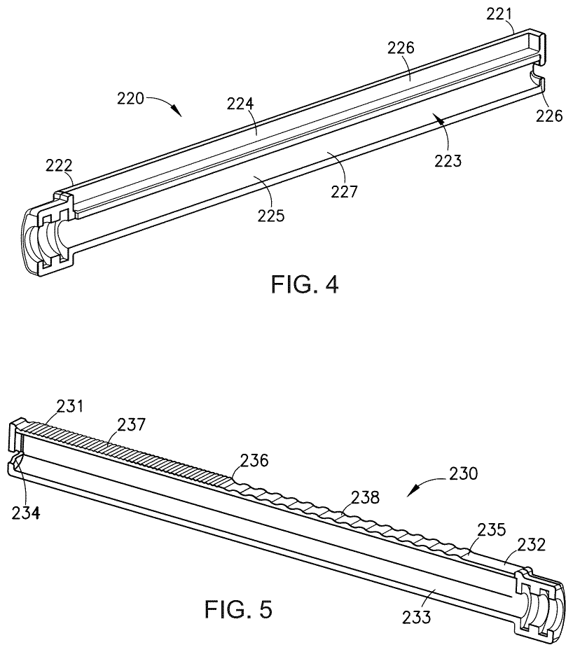

According to embodiments of the disclosure, each support insert 300 may have a cross-sectional shape that is based at least in part on a cross-sectional shape of the second portion 215 of the inner volume 213 defined by the introducer 210, for example, a circular cross-sectional shape. In this manner, the inner surface 223 of the first member 220 (FIG. 4) and the inner surface 233 of the second member 230 (FIG. 5) can support and/or guide the support insert 300 as the support insert 300 is moved relative to the introducer 210, as explained in further detail below. The support insert 300 includes an opening 302 formed therethrough, such as at a center point thereof, that is sized and shaped to allow the catheter 260 to freely pass therethrough, while being contained thereby to prevent excessive movement of the catheter 260 within the opening 288. In some embodiments, the support insert 300 may be formed of a moldable material that provides for easy manufacturing thereof, with the support insert 300 being formed of a polymeric material that may be injection molded, as a non-limiting example.

Referring now to FIGS. 13-16, and with continued reference to FIGS. 1-12, changing of the blood draw device 200 from the first configuration to the second configuration is shown. The blood draw device 200 can be in the first configuration prior to use and can be transitioned by a user (e.g., a doctor, physician, nurse, technician, phlebotomist, and/or the like) from the first configuration to the second configuration to dispose at least the distal end portion 262 of the catheter 260 in a distal position relative to the housing 218 (e.g., within/pas the distal tip of indwelling catheter 312 (FIG. 1)).

Referring first to FIG. 13, the blood draw device 200 is shown in the first configuration when the catheter 260 and support tube 280 are disposed in the first catheter position and the first tube position, respectively within the housing 218. In some embodiments, substantially the entire catheter 260 and support tube are disposed within the housing 218 when in their respective first positions. In such embodiments, the housing 218 can include a seal or the like (as described above) that can substantially seal the distal end portion 212 of the housing 210 to isolate the catheter 260 and support tube 280 within the second portion 215 of the inner volume 213. In other embodiments, the catheter 260 and support tube 280 are disposed within the housing 210 and the lock 240 when in their respective first positions.

With the blood draw device 200 in the first configuration, the primary advancement member 270 is disposed toward the proximal end portion 211 of the introducer 210 and the secondary advancement member 290 is disposed somewhat centrally on the introducer 210. The proximal end portion 261 of the catheter 260 is coupled to the second portion 275 of the advancement member 270, and the catheter 260 extends through the second portion 215 of the inner volume 213 of the introducer 210 and through the passageway 285 of each of the catheter support inserts 300 (such as the two support inserts 300 shown in FIGS. 13-16), with the distal end portion 262 of the catheter 260 positioned within the support tube 280. The proximal end portion 281 of the support tube is coupled to the second portion 295 of the secondary advancement member 290, and the support tube 280 extends through the second portion 215 of the inner volume 213 of the introducer 210, so as to be received in the lumen of the coupler 216 and/or the lumen 255 of the lock 240.

With the blood draw device 200 in the first configuration, the catheter support inserts 300 are positioned in their initial pre-use position, such as being spaced apart equidistantly along the unsupported portion of the catheter 260 (extending from the second portion 275 of the primary advancement member 270 to the second portion 295 of the secondary advancement member 290). In some embody (not shown) that are formed on the inner surface of the introducer housing (i.e., inner surface 223 of first housing member 220). The bump projections protrude inwardly from the inner surface 223 of the housing 218 and may be formed at locations thereon corresponding to the pre-use positions of the catheter support inserts 300.

To being the transition of the blood draw device 200 from the first configuration to the second configuration, the user first engages the engagement member 292 of the secondary advancement member 290 to move the advancement member 290 relative to the housing 218, which in turn, moves the support tube 280 from the first tube position (e.g., disposed within the housing 218) toward the second tube position. In this manner, the support tube 280 is moved through the second portion 215 of the inner volume 213 and the lumen 255 of the lock 240 and is advanced into and through the VAD 310, such that the distal end 282 of the support tube 280 extends out past the indwelling catheter 312 and into the vein of a patient. As explained previously, the support tube 280 is formed of a kink-resistant material such as a shape memory alloy (e.g., Nitinol), which allows the support tube 280 to advance through the VAD 310 without fear of kinking.

After movement of the secondary advancement member 290 in the distal direction, the blood draw device 200 is considered to be in an intermediate configuration as shown in FIG. 14, with the support tube 280 in the second tube position (advanced through VAD 310) but the catheter 260 still in the first catheter position (within housing 218 of introducer 210).

Upon the blood draw device 200 being actuated to the intermediate configuration, a user may then engage the engagement member 272 of the primary advancement member 270 to move the advancement member 270 relative to the housing 218, thereby transitioning the blood draw device 200 from the intermediate configuration to the second configuration. Actuation of the primary advancement member 270 distally along the housing 218 moves the catheter 260 from the first catheter position (e.g., disposed within the housing 218) toward the second catheter position, as shown in FIG. 15. As the catheter 260 is moved distally, the catheter 260 advances through the second portion 215 of the inner volume 213 and the lumen 255 of the lock 240 and advances into and through the VAD 310, such that the distal end 282 of the catheter 260 extends out past the indwelling catheter 312 and out past the support tube 280, and into the vein of the patient. As described above, with the support tube 280 already moved to its second tube position, advancement of the catheter 260 to the second catheter position may be performed without fear of the catheter 260 kinking, as the catheter 260 is advanced distally through the support tube 280, which is positioned coaxially thereabout. Due to the inner diameter of the lumen 283 of support tube 280 being only slightly larger than the outer diameter of the catheter 260, and due to the anti-kinking properties of the support tube 280, the support tube 280 functions to guide the catheter 260 and preventing kinking thereof as the catheter is distally advanced to the second catheter position.

In some embodiments, as the primary advancement member 270 is moved distally along the housing 218 to transition the blood draw device 200 from the intermediate configuration to the second configuration, the advancement member 270 contacts a proximal-most catheter support insert 300a. As the advancement member 270 is advanced further in the distal direction, the advancement member 270 pushes the proximal-most catheter support insert 300a in the distal direction. According to aspects of the disclosure, the pushing of the catheter support insert 300a by the advancement member 270 is sufficient to force the catheter support insert 300a past a bump projection on the housing 218 that held the catheter support insert 300a in its pre-use position. Continued advancement of the primary advancement member 270 in the distal direction causes the proximal-most catheter support insert 300a to eventually come into contact with the next catheter support insert 300b positioned on the catheter 260. With the proximal-most catheter support insert 300a brought into contact with the next catheter support insert 300b and the primary advancement member 270 continuing to advance, the advancement member 270 and catheter support insert 300a push the next catheter support insert 300b in the distal direction (past its engagement with a bump projection). In some embodiments, upon the blood draw device 200 reaching the second configuration, the catheter support inserts 300a, 300b may be moved into contact with the second portion 295 of secondary advancement member 290, while in other embodiments, a gap may remain between the catheter support inserts 300a, 300b and the second portion 295 of secondary advancement member 290.

With the blood draw device 200 in the second configuration, the catheter 260 is thus moved to its second position, where the distal end portion 262 thereof is placed in a desired position relative to a distal end of the indwelling catheter 312, as shown in FIG. 16. In some instances, for example, a distal end 262 of the catheter 260 can extend a predetermined distance beyond the distal end of the indwelling catheter 312, such that the distal end 262 of the catheter 260 is positioned within the vein at a predetermined distance beyond the distal surface of the catheter 260 (e.g., a position within a vein that is substantially free from debris (e.g., fibrin/blood clots) otherwise surrounding the distal end portion of the indwelling catheter 312).

In some embodiments, with the catheter 260 in the second catheter position, the user can retract the support tube 280 from its second tube position prior to performing of a blood draw. That is, a user may user may push the secondary advancement member 290 proximally along the housing 218, in order to withdraw the support tube 280 back into the indwelling catheter 312, in order to optimize conditions for the blood draw.

With the catheter 260 in the second catheter position (and the support tube 280 optionally retracted back proximally), the user can establish fluid communication between a fluid reservoir, fluid source, syringe, and/or the like and the catheter 260. With the catheter 260 in fluid communication with the fluid reservoir and/or fluid source, the blood draw device 200 can then aspirate a volume of blood from the vein based at least in part on disposing the distal surface of the catheter 260 at the predetermined and/or desired distance beyond the distal tip of the indwelling catheter 312. In some instances, once a desired amount of blood has been collected and/or once a desired volume of a drug has been delivered to the patient, the user can move the primary advancement member 270 in the proximal direction, such as moving the advancement member 270 back to its proximal most position, so as to move the catheter 260 back to the first position.

In this manner, blood draw device 200 may be operated to advance catheter 260 into the indwelling catheter 312 while minimizing the potential for bending, kinking, or deformation of the catheter 260.

Referring now to FIGS. 17-19, it is recognized that some embodiments of blood draw device 200 may further incorporate one or more sensors and accompanying electronics/circuitry into the support tube 280 and the secondary advancement member 290, in order to further facilitate advancement of the catheter 260 into/through the VAD 310. That is, readings from the sensor may help determine whether it is appropriate to advance the catheter 260 to the second catheter position, for performing of a blood draw, or if there are problems with the indwelling catheter 312.

Referring first to FIG. 17, support tube 280 is shown as including a sensor 320 incorporated into the distal end portion 282 thereof, with the sensor 320 embedded into the support tube 280 or formed on the external surface thereof.

In one embodiment, the sensor 320 may comprise a pressure sensor that is embedded in the support tube 280 and is operable to evaluate a line pressure within the indwelling catheter 312 as the support tube 280 is advanced toward the second tube position. Detection of a pressure difference by the sensor 320 may be used to indicate if the support tube 280 exited the indwelling catheter 312, such that subsequent advancement of the catheter 260 may be performed, or may be used to indicate that the indwelling catheter 312 of VAD 310 is occluded, such that insertion of catheter 260 is not appropriate.

In another embodiment, the sensor 320 may comprise a near infrared (NIR) sensor that is mounted externally on the support tube 280 and that uses light transmission and absorption to measure various physiological parameters at the site of the indwelling catheter 312 insertion. Upon the sensor 320 advancing out of the indwelling catheter 312 and into the patient vein, the sensor 320 may give an indication of its location, based on a measured transmission of light diagonally across the support tube internal diameter (confirming a blood flow into the support tube 280). Conversely, if there is an occlusion of the indwelling catheter 312, the sensor 320 may determine the presence of such an occlusion based on a measured transmission of light diagonally across the support tube internal diameter.

Referring now to FIG. 18, support tube 280 is shown as including a flow sensor 320 incorporated into the distal end portion 282 thereof, with the flow sensor being housed in a split septum 322 provided at the distal tip of the support tube 280. The flow sensor 320 operates to detect a change in flow rate of a fluid into the support tube 280, thereby indicating clinical conditions in the VAD 310, such as partial line occlusion in the indwelling catheter 312 due to thrombus build-up. Due to the split septum 322, passage of the catheter 260 through the distal end portion 282 of support tube 280 is still enabled.

In any of the sensor embodiments of FIGS. 17 and 18, the sensor 320 may be in operable communication with an electronics module 324 incorporated into the secondary advancement member 290, as illustrated in FIG. 19. That is, the secondary advancement member 290 may include an electronics module 324 incorporated therein that comprises a communication module 326 operatively connected to the sensor 320 to receive signals therefrom regarding acquired operational data, along with an integrated circuit 328 configured to process the signals received by the communication module 326, and an indicator 330 controlled by the integrated circuit 328 and configured to generate an audio or visual alert responsive to the acquired operational data. In this manner, the electronics module 324 may relay and indicate to the user, conditions of the VAD 310 (i.e., indwelling catheter 312) and/or whether it is appropriate to advance the catheter 260 into/through the indwelling catheter 312 for performing of a blood collection, including when the support tube 280 and catheter 260 have been advanced to a proper location inside the vasculature. As further shown in FIG. 19, a flexible clip 332 may be provided that secures the electronics module 324 to the secondary advancement member 290.

Beneficially, embodiments of the disclosure are directed to a blood draw device that provides for advancement of an extension catheter into an indwelling catheter of a VAD while minimizing the potential for bending, kinking, or deformation of the catheter. The blood draw device includes a support tube within the introducer housing that may be advanced into the VAD prior to advancement of the extension catheter. The support tube is formed of a shape memory alloy or other suitable material with anti-kinking properties, so that the support tube may initially be advanced into the patient vasculature without kinking thereof. The catheter may then subsequently be advanced through the support tube, with the support tube guiding the catheter and preventing kinking thereof, so that the catheter may be placed into the patient vasculature at a location ideal for blood collection.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A blood draw device for advancing a catheter into an indwelling catheter of a vascular access device, the blood draw device comprising:
an introducer comprising a housing having a proximal end portion and a distal end portion, with the housing defining an inner volume;
a coupling member positioned at the distal end portion and coupleable to the vascular access device;
a catheter movably received within the inner volume;
a support tube movably received within the inner volume and defining a tube lumen therein configured to receive the catheter, with the support tube formed of a flexible, kink-resistant material;
a primary advancement member movably coupled to the housing and configured to move relative to the housing to move the catheter between a first catheter position, in which the catheter is disposed within the introducer or within the introducer and the coupling member, and a second catheter position, in which a distal end portion of the catheter is disposed beyond the distal end portion of the introducer and into or distally past a distal end of the indwelling catheter; and
a secondary advancement member movably coupled to the housing, distal from the primary advancement member, and configured to move relative to the housing to move the support tube between a first tube position, in which the support tube is disposed within the introducer or within the introducer and the coupling member, and a second tube position, in which a distal end portion of the support tube is disposed beyond the distal end portion of the introducer and into or distally past a distal end of the indwelling catheter;
wherein, with the support tube in the second tube position and the catheter moving from the first position to the second catheter position, the support tube is arranged coaxially about a portion of the catheter, to prevent kinking of the catheter as it is advanced from the first catheter position to the second catheter position.

2. The blood draw device of claim 1, wherein the support tube is formed of a shape memory alloy.

3. The blood draw device of claim 2, wherein the shape memory alloy comprises Nitinol.

4. The blood draw device of claim 1, wherein the primary advancement member and the secondary advancement member are arranged on the housing such that movement of the catheter from the first catheter position to the second catheter position, via distal movement of the primary advancement member along the housing, is enabled only after movement of the support tube from the first tube position to the second tube position, via distal movement of the secondary advancement member along the housing.

5. The blood draw device of claim 1, further comprising one or more catheter support inserts positioned within the inner volume and positioned between the primary advancement member and the secondary advancement member, each of the one or more catheter support inserts receiving the catheter to provide support to a length of the catheter that extends between the primary advancement member and the secondary advancement member.

6. The blood draw device of claim 5, wherein for a respective catheter support insert of the one or more catheter support inserts that is nearest the primary advancement member, the primary advancement member engages the catheter support insert during movement thereof when moving the catheter from the first catheter position to the second catheter position, with the primary advancement member engaging and moving the catheter support insert with respect to the housing.

7. The blood draw device of claim 5, wherein the one or more catheter support inserts comprises a plurality of catheter support inserts spaced apart along a length of the catheter between the primary advancement member and the secondary advancement member.

8. The blood draw device of claim 1, wherein the support tube comprises a sensor positioned at a distal end of the support tube and this is configured to acquire operational data when advancing the support tube from the first tube position to the second tube position.

9. The blood draw device of claim 8, wherein the sensor comprises a pressure sensor configured to evaluate a line pressure within the indwelling catheter, as the support tube moves from the first tube position to the second tube position.

10. The blood draw device of claim 8, wherein the sensor comprises a near infrared (NIR) sensor configured to provide positional data for the support tube relative to the indwelling catheter and/or to detect an occlusion within the indwelling catheter.

11. The blood draw device of claim 8, wherein the sensor comprises a flow sensor configured to detect a flow rate of fluid into the indwelling catheter.

12. The blood draw device of claim 11, wherein the flow sensor is housed on a split septum valve at the distal end of the support tube, the split septum valve allowing for passage of the catheter therethrough when the catheter is moved to second catheter position.

13. The blood draw device of claim 8, wherein the secondary advancement member comprises an electronics module incorporated therein, the electronics module comprising:
a communication module operatively connected to the sensor to receive signals therefrom regarding the acquired operational data;
an integrated circuit configured to process the signals received by the communication module; and
an indicator controlled by the integrated circuit and configured to generate an audio or visual alert responsive to the acquired operational data.

14. The blood draw device of claim 1, wherein the support tube comprises scale markings thereon indicating a length by which the support tube has been distally advanced as it moves from the first tube position to the second tube position.

15. The blood draw device of claim 1, wherein each of the primary advancement member and the secondary advancement member comprises:
a first portion comprising an engagement portion movable along a top surface of the housing; and
a second portion positioned at least partially within the inner volume and joined to the first portion through a slot formed in the top surface of the housing;
wherein, for the primary advancement member, the second portion is coupled with a proximal end of the catheter; and
wherein, for the secondary advancement member, the second portion is coupled with a proximal end of the support tube.

16. The blood draw device of claim 15, wherein each of the primary advancement member and the secondary advancement member slide within the slot formed in the top surface of the housing, to move relative to the housing,

17. The blood draw device of claim 1, wherein the coupling device comprises a lock including:
a coupler configured to engage the distal end portion of the housing;
a first arm and a second arm configured to engage with the vascular access device or an adapter provided between the vascular access device and the lock; and
a blunt cannula extending from the coupler and disposed between the first arm and the second arm, the blunt cannula configured to engage with the vascular access device or the adapter;
wherein a lumen extends through the blunt cannula and the coupler, through which the support tube and catheter may be distally advanced.

18. A blood draw device for advancing a catheter into an indwelling catheter of a vascular access device, the blood draw device comprising:
an introducer comprising a housing having a proximal end portion and a distal end portion, with the housing defining an inner volume, and with the housing including a slot formed therein;
a primary advancement member movably coupled to the housing and configured to slide within the slot of the housing;
a secondary advancement member movably coupled to the housing and configured to slide within the slot of the housing, the secondary advancement member positioned distal from the primary advancement member;
a catheter movably received within the inner volume, the catheter comprising a distal end and a proximal end, with the proximal end secured to the primary advancement member, such that movement of the primary advancement member causes a corresponding movement of the catheter; and
a support tube movably received within the inner volume and defining a tube lumen therein configured to receive the catheter, the support tube comprising a distal end and a proximal end, with the proximal end secured to the secondary advancement member, such that movement of the secondary advancement member causes a corresponding movement of the support tube;
wherein, with each of the primary advancement member and the secondary advancement member moved to a respective distal-most position on the housing, a portion of each of the catheter and the support tube is disposed beyond the distal end portion of the introducer, with the catheter being advanced into and through the support tube to prevent kinking of the catheter as it is distally advanced.

19. The blood draw device of claim 18, wherein the support tube is formed of a shape memory alloy that resists kinking when the support tube is advanced distally into the indwelling catheter of the vascular access device, via movement of the secondary advancement member.

20. The blood draw device of claim 18, further comprising one or more catheter support inserts positioned within the inner volume and positioned between the primary advancement member and the secondary advancement member, each of the one or more catheter support inserts receiving the catheter to provide support to a length of the catheter that extends between the primary advancement member and the secondary advancement member.
